(19) United States
(12) Patent Application Publication
(10) Pub. No.: US 2021/0388120 A1
(43) Pub. Date: Dec. 16, 2021

(54) PRODUCTION OF BIOMEDICAL COMPOUNDS BY ENRICHMENT CULTURES OF MICROORGANISMS

(71) Applicant: TECHNISCHE UNIVERSITEIT DELFT, Delft (NL)

(72) Inventors: Marinus Cornelis VAN LOOSDRECHT, Delft (NL); Yuemei LIN, Delft (NL); Martin PABST, Delft (NL); Hugobert Bernhard Crijn KLEIKAMP, Delft (NL)

(73) Assignee: TECHNISCHE UNIVERSITEIT DELFT, Delft (NL)

(21) Appl. No.: 17/288,463

(22) PCT Filed: Oct. 23, 2019
(86) PCT No.: PCT/NL2019/050696

§ 371 Date: Apr. 23, 2021

(30) Foreign Application Priority Data
Oct. 25, 2018 (NL) 2021875

(51) Int. Cl. C08B 37/00; C12N 1/20; C12P 19/26; C12P 21/00; C12P 19/12; C12P 19/02

(52) U.S. Cl. CPC C08B 37/0075; C12N 1/20; C12P 19/02; C12P 21/005; C12P 19/12; C12P 19/26

(57) ABSTRACT

The present invention is in the field of a method for production of biomedical compounds by enrichment cultures of microorganisms, and a product obtainable by said methods. The microorganisms are grown in a batch reactor, a continuous reactor, a semi-continuous reactor, such as a Nereda® reactor.

This application is a national entry of International Patent Application PCT/NL2019/050696, filed Oct. 23, 2019, in the name of “TECHNISCHE UNIVERSITEIT DELFT”, which PCT-application claims priority to Netherlands Patent Applications with Serial No. 2021875, filed Oct. 25, 2018, in the name of “TECHNISCHE UNIVERSITEIT DELFT”. The entire contents of the above-referenced applications and of all priority documents referenced in the Application Data Sheet filed herewith are hereby incorporated by reference for all purposes.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### FIELD OF THE INVENTION

The present invention is in the field of a method for production of biomedical compounds by enrichment cultures of microorganisms, and a product obtainable by said methods. The microorganisms are grown in a batch reactor, a continuous reactor, a semi-continuous reactor, such as a Nereda® reactor.

### BACKGROUND OF THE INVENTION

The present invention is in the field of production of biomedical compounds by microorganisms, specifically bacteria. Biomedical sciences relate to applied sciences wherein natural science and formal science as well as technology are applied for use in healthcare or public health. The field of biomedical sciences relates therefore to life and natural sciences, such as (medical) microbiology, and biomedical engineering. The present invention relates to microorganisms that are grown in a reactor.

A biomedical compound considered is heparin. Heparin (CAS Number 9005-49-6) is a naturally occurring anticoagulant produced by basophils and mast cells. Prior to 1933, heparin was available in small amounts, was extremely expensive and toxic, and, as a consequence, of no medical value.

Pharmaceutical-grade heparin is nowadays derived from mucosal tissues of slaughtered animals such as pig intestines and cattle lungs. Some advances to produce heparin synthetically have been made since 2003 but the vast majority of heparin is still produced from the above tissues. Although recently a chemoenzymatic process of synthesizing low molecular weight heparins from simple disaccharides was reported, said process is still not very relevant in terms of quantities. Because of its structural complexity, the shortage of raw materials, and the numerous synthetic steps combined with low product yields, production of heparine and its oligosaccharides via chemical methods is very difficult.

Oduah discusses several strategies for producing heparin including microbial production, mammalian cell production, and chemoenzymatic modification (Pharmaceutical 2016, Vol. 9, 38, doi:10.3390/ph9030038). Monodisperse heparinoids have been developed using E. coli K5, which is a natural producer of the polysaccharide heparosan, an unsulfated “precursor” of heparin and HS produced in eukaryotic cells. The initial studies using this system were not favorable, though some further advances have been made by conversion of the product formed using recombinant strategies. A further report relates to E. coli K5 that is used for biosynthesis of heparosan which is initiated on 2-keto-3-deoxyoculosonic acid glycolipid acceptor (Vaidyanathan in Bioengineering&Translated Medicine 2017, 2, p. 17-30). Further enzymatic action may be used to elongate the heparosan, typically by introducing a gene or the like in the microorganisms, typically in an iterative time consuming and expensive biochemical synthesis. Also Zhang (Chem. Sci. 2017, 8, 7932) show similar advances by chemoenzymatic synthesis.

Recently Kang (CellPress Reviews, Trends in Biotechnology, August 2018, Vol. 38, No. 8, p. 806-818) reported advances in production of molecules as glycosaminoglycans (GAG), such as hyaluronan, by microorganisms. Such is typically done by introducing specific genes into specific microorganisms. Therein use is made of biochemical pathways. They conclude that biosynthesis of GAGs and their oligosaccharides using microbial and enzymatic approaches has progressed, but that hurdles still have to be taken. The focus remains of engineering microorganisms.

Native heparin is a polymer with a molecular weight ranging from 3 to 30 kDa, whereas commercial heparin is in the range of 12 to 15 kDa. Heparin is a member of the glycosaminoglycan family of carbohydrates (which includes the closely related molecule heparan sulfate) and consists of a variably sulfated repeating disaccharide unit. The most common disaccharide unit is composed of a 2-O-sulfated iduronic acid and 6-O-sulfated, N-sulfated glucosamine, IdoA(2S)—GlcNS(6S).

Heparin, also known as unfractionated heparin (UFH), is used as an anticoagulant (blood thinner). Specifically it is used to treat and prevent deep vein thrombosis, pulmonary embolism, and arterial thromboembolism. It is also used in the treatment of heart attacks and unstable angina. It is given by injection into a vein. Other uses include coatings for test tubes and kidney dialysis machines.

Sialic acid is a generic term for the N- or O-substituted derivatives of neuraminic acid, a monosaccharide with a nine-carbon backbone (CAS nr. 114-04-5). It is also the name for the most common member of this group, N-acetylneuraminic acid (Neu5Ac or NANA). The sialic acid family includes more than 50 derivatives of neuraminic acid. Unsubstituted neuraminic acid are not found in nature. The amino group of neuraminic acid is typically substituted either by an acetyl or glycolyl residue, and the hydroxyl groups may be methylated or esterified with sulphate, phosphate, acetyl, or lactyl groups, and combinations thereof. The carboxylate group of sialic acid may result in a net negative charge. Therewith cations, such as Ca²⁺, can be bound, which may contribute to the stability of aerobic granular sludge. This charge and the sugar hydroxyl groups may support binding of water molecules. Sialic acid is typically synthesized by glucosamine 6 phosphate and acetyl-CoA through a transferase, resulting in N-acetylglucosamine-6-P, converted into N-acetylmannosamine-6-P, which produces N-acetylneuraminic-9-P (sialic acid) by reacting with phosphoenolpyruvate. In bacterial systems, sialic acids are biosynthesized by an aldolase enzyme. The enzyme uses a mannose derivative as a substrate, inserting three carbons from pyruvate into the resulting sialic acid structure. These enzymes can be used for chemoenzymatic synthesis of sialic acid derivatives.

Sialic acid-rich glycoproteins bind selectin. Many bacteria that live in association with higher animals use sialic acid derived from their host. Many of these incorporate sialic acid into cell surface features like their lipopolysaccharide and capsule, which helps them evade the innate immune response of the host. Other bacteria simply use sialic acid as a good nutrient source, as it can be converted to fructose-6-phosphate, which can then enter central metabolism.

Aerobic granular sludge (AGS) is an upcoming technology for wastewater treatment, capable of simultaneously removing organic carbon, nitrogen, and phosphorus typically in a single process unit. The sludge granules consist of bacteria encapsulated in a matrix of extracellular polymeric substances (EPS). Besides providing a structural matrix in which cells can grow, EPS may also serve as a protection against adverse conditions in the bulk liquid. EPS are found in all kinds of sludge, and a multitude of properties and compositions have been described with different operating conditions. Examples of such production methods can be found in WO2015/057067 A1, and WO2015/050449 A1, whereas examples of extraction methods for obtaining said biobased polymers can be found in Dutch Patent application NL2016441 and in WO2015/190927 A1. Specific examples of obtaining these extracellular substances, such as aerobic granular sludge, and anammox granular sludge, and the processes used for obtaining them are known from Water Research, 2007, for anammox granular sludge doi:10.1016/j.waters.2007.03.044 and for aerobic granular sludge Water Science and Technology, 2007, 55(8-9), 75-81. Further, Li et al. in Water Research, Elsevier, Amsterdam, NL, Vol. 44, No. 11 (Jun. 1, 2010), pp. 3355-3364) recites specific alginates in relatively raw form. Details of the biopolymers can also be found in these documents, as well as in Dutch Patent applications NL2011609, NL2011542, NL2011852, and NL2012089. These documents, and their contents, are incorporated by reference. Granular sludge generally does not contain a significant fraction of pathogens.

Dirac et al, in Water Science and Technology, Vol. 38, no. 8-9, p. 45-53, 1998, recites growth, isolation, and extraction of extracellular polymers produced in activated sludge. The polymers comprise polysaccharides. The saccharides identified are (see table 3) e.g. rhamnose, mannose, galactose, and mainly glucose. None of these is considered to be a biomedical compound, nor is any specific biomedical compound mentioned. Ortega et al, in J. Applied Microbiology, Vol. 102, No. 1, 2007, p. 254-264, recites characterization of extracellular polymers from biofilm bacteria. In one example a glycoprotein is found, in another a polysaccharide with uronic acid and hexosamine Table 2 shows presence of mainly neutral sugars, and smaller amounts of hexuronic acids and hexosamines. Table 3 shows low amounts of monosaccharides, such as Glc-N—Ac. WO2008/042975 A2 recites compositions, methods and system for making and using cyanobacteria that produce extracellular saccharides, such as for the manufacture of cellulose and saccharides, CO2 fixation, the production of alternative sources of cellulose and saccharides for conventional applications, as well as for biofuels and precursors thereof. U.S. Pat. No. 4,966,845 (A) recites a nonnutritive sweetener L-altrose which is obtained from extracellular polysaccharides elaborated by certain strains of the bacterium Butyrivibrio fibrisolvens when grown on a carbohydrate-containing nutrient medium. L-altrose has not previously been found in nature. WO2015/190927 (A1) relates to a reactor set up wherein dense aggregates of microorganisms are formed, typically in or embedded in an extracellular matrix. Albeit the dense aggregates comprising extracellular polymeric substances, or biopolymers, in particular linear poly-saccharides, these do not relate to the present invention.

The present invention therefore relates to an improved method for producing glycosaminoglycans and sialic acids, and products obtained, which solve one or more of the above problems and drawbacks of the prior art, providing reliable results, without jeopardizing functionality and advantages.

### SUMMARY OF THE INVENTION

The present invention relates to an improved method of production of a biomedical compound. Inventors grow bacteria in granular form, such as in seawater-adapted aerobic granular sludge (AGS). In a reactor set up a (non-axenic) bacteria culture may be fed with a suitable carbon sources, in an aqueous environment. Therein dense aggregates of microorganisms are formed, typically in or embedded in an extracellular matrix, which matrix contains extracellular polymeric substances (EPS). Such may relate to granules, to sphere like entities having a higher viscosity than water, globules, a biofilm, etc. Granules making up granular sludge are (dense) aggregates of microbial cells self-immobilized through extra-cellular polymeric substances into a spherical form without any involvement of carrier material. A characterizing feature of granules of granular sludge is that they do not significantly coagulate during settling (i.e. in a reactor under reduced hydrodynamic shear). Extracellular polymeric substances make up a significant proportion of the total mass of the granules. The granular sludge may comprise granules in a size range of about 0.2-15 mm, preferably 0.5-5 mm, such as 1.0-3 mm. A size of about 0.2 mm (more precise 0.212 mm) is regarded to be a minimum size to consider sludge to be granular. Advantageously, granules of granular sludge can be readily removed from a reactor by e.g. physical separation, settling, centrifugation, cyclonic separation, decantation, filtration, or sieving to provide extracellular polymeric substances in a small volume. Compared to separating material from a liquid phase of the reactor this means that neither huge volumes of organic nor other solvents (for extraction), nor large amounts of energy (to evaporate the liquid) are required for isolation of the extracellular substances. For various applications the extracellular compounds, in this document also referred to as biomedical compounds, can not be used directly, e.g. in view of insufficient purity (typically >50%), a coloring of the extracellular substances, etc. If the quality, e.g. in terms of purity, needs to be further improved (e.g. >90%, or even >99%) in view of intended applications, standard chemical techniques can be use thereto.

In the method slow growing carbon accumulating microorganisms, such as PAOs (poly-phosphate accumulating organisms) and GAOs (glycogen accumulating organisms), are favoured. The term “slow” indicates that a weight of a culture increases by less than 30% weight/day, preferably less than 20% weight/day, such as less than 10% weight/day. As a result the culture is enriched with these species. Such can be achieved by switching at least once between aerobic and anaerobic conditions, preferably in a cyclic mode. Often the cycles are repeated, such as 2-10 times. In these cycles aeration typically takes place during a period of 30 minutes to 12 hours, such as 60 minutes to 6 hours. After growing and forming the extracellular matrix the biomass can be harvested, such as by physically separating, and the extracellular matrix being separated from the microbial cell substances. From the harvested material the biomedical compound can be extracted from the extracellular matrix. The biomedical compound of the present invention comprises a monosaccharide or disaccharide, or a salt thereof, or conjugate thereof, or a combination thereof, and is selected from (i) at least one of a heparan like polymer, a heparin like compound, and a heparin oligomer, and from (ii) at least one of a neuraminic acid glycosaminoglycan, a sialic acid, a glycoprotein, and a glycolipid, such as a glycosaminoglycan, preferably a 3-30 kDa glycosaminoglycan, such as a 12-15 kDa glycosaminoglycan, a sialic acid, a glycoprotein, a glycolipid, a combination thereof, such as a sialoglycoprotein, depending e.g. on the method of extracting. An example of a structural formula is

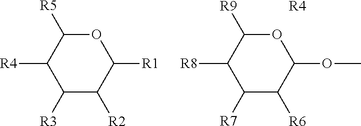

wherein R1-R9 are as explained below (R4 being the right most structure). Typically 0.1-30 wt. % of (i) a heparan like polymer, a heparin like compound, and a heparin oligomer is/are present, preferably 0.5-20 wt. %, more preferably 1-18 wt. %, even more preferably 2-15 wt. %, such as 5-12 wt. %, and (ii) 0.1-30 wt. % of a neuraminic acid glycosaminoglycan, a sialic acid, a glycoprotein, and a glycolipid is/are present, preferably 0.5-20 wt. %, more preferably 1-18 wt. %, even more preferably 2-15 wt. %, such as 5-12 wt. %, wherein all wt. % are based on a dry weight of the extracellular matrix.

It is now been found that the material which forms the extracellular matrix in which microorganism cells are embedded contains relatively high amounts of heparan/heparin like polymers and sialic acids. Test results indicate presence of these compounds. By growing these bacteria e.g. on waste water or pure substrate these compounds are harvested. The bacteria can be grown in reactors under controlled conditions and if required supplemented with a defined medium, which is considered preferential for biomedical applications. The medium may comprise at least one of sugars, fatty acids, proteins, proteins, and minerals. As such high amounts of these compounds can be produced in good quality.

With the term “microbial process” here a microbiological conversion is meant.

So it has been found that components of the EPS may be sialoglycoproteins (1-6 wt. %) and that most likely it is at least Ca. Accumulibacter in seawater-adapted AGS that contains genes that encode for enzymes that are responsible for sialic acid metabolism.

Inventors therefore have provided a method for obtaining sialic acid in aerobic granular sludge, such as in seawater-adapted AGS. Presence of sialic acid is shown on both the bacterial cell surface and in the EPS matrix. Most likely the sialic acids have an α2-3 (NANA(α2-3) Gal, implying a galactose linkage) or α2-6 linkage (GlcNAc(β1-4) GlcNAc, implying a linkage with GlcNAc within the EPS matrix).

Details of the present invention in respect of sialic acids and so on may be found in a to be published paper by Lin et. al. entitled “Sialic Acids Bound to Glycoproteins in the Extracellular Polymeric Substances of Seawater-Adapted Aerobic Granular Sludge” which document and its contents are incorporated by reference herein.

Inventors have also found (sulphated) glycosaminoglycan (sGAG) being present in Anammox and Aerobic Granular Sludge (AGS) Extracellular Polymeric Substances (EPS). Extracted EPS has been tested using a commercially available sGAG assay (Blyscan) using dimethyl-methylene blue (DMMB). The EPS has been pre-treated before analyzing. These pre-treatments had a positive effect on the apparent sGAG content of Anammox EPS. Initially finding 1.21% sGAG content, digestion found 2.47% and an additional denaturation step yielded 2.65% (w/w) in annamox sludge and 6.67% sGAG in AGS.

In a second aspect the present invention relates to a product obtained by the method according to the invention. As the present product is of biological origin, and produced by microorganisms, the composition thereof is by definition different from e.g. a chemically obtained product, or otherwise obtained product. Such is e.g. evidenced by trace compounds of the microorganism culture used, such as DNA, and proteins thereof, as well as an exact chemical structure, as well as a distribution of biomedical compounds, such as presence of 5 kDa, 8 kDa and 12 kDa compounds.

In a third aspect the present invention relates to a method of transferring genes from a slow growing PAOs (poly-phosphate accumulating organisms) and GAOs (glycogen accumulating organisms) according to the invention, comprising identifying one or more genes in a biochemical pathway for producing a glycosaminoglycan, preferably a 3-30 kDa glycosaminoglycan, such as a 12-15 kDa glycosaminoglycan, a sialic acid, a glycoprotein, a glycolipid, isolating the one or more genes, transferring the isolated genes to a suitable host, such as E. coli, preferably using a CRISPR technique. Therewith yield and purity can be improved. A metagenome analysis was performed on the major species that were found in this sludge, to screen for putative production pathways for sialic acids.

Thereby the present invention provides a solution to one or more of the above mentioned problems and drawbacks.

Advantages of the present description are detailed throughout the description.

### DETAILED DESCRIPTION OF THE INVENTION

The present invention relates in a first aspect to a method producing a biomedical compound according to claim 1.

In an exemplary embodiment of the present method the compound may be at least one of a heparan like polymer, a heparin like compound, a heparin oligomer, or a neuraminic acid.

In an exemplary embodiment the monosaccharide or disaccharide has the above structural formula (see also FIG. 4a), wherein each of R1-R9 is independently selected, wherein R1 is selected from at least one of H, COOH, and OH, wherein R2 is selected from at least one of NHAc, NHSO₃H, and H, wherein R3 is selected from at least one of H, and OH, wherein R4 is selected from at least one of H, NHAc, and OH, and for the disaccharide R4 has the above structural formula (see also FIG. 4b), wherein R6 is selected from at least one of H, OSO₃H, and OH, wherein R7 is selected from at least one of OH, and H, wherein R8 is selected from at least one of H, and OH, wherein R9 is selected from at least one of H, COOH, and OH, and wherein R5 is selected from at least one of H, CH₂OH, CH₂OSO₃, COOH, CHOHCH₂OH, CH₂CHOHCH₂OH, and OH.

In an exemplary embodiment of the present method the microorganisms may be grown in granules.

In an exemplary embodiment of the present method the microorganisms may be grown under aerobic conditions, form aerobic granular sludge, or wherein the microorganisms are grown under aerobic and anaerobic conditions by switching at least once between aerobic and anaerobic conditions, preferably in a cyclic mode.

In an exemplary embodiment of the present method the compound may be a 5-15 kDa glycosaminoglycan, such as a 5 kDa, 8 kDa and 12 kDa glycosaminoglycan, such as a sulphated glycosaminoglycan, such as at least one of a heparan like polymer, a heparin like compound, a heparin oligomer, or a neuraminic acid.

In an exemplary embodiment of the present method the compound may be a sialic acid, such as N-acetylneuraminic acid (Neu5Ac).

In an exemplary embodiment of the present method the microorganisms may be provided with a supplement.

In an exemplary embodiment of the present method the microorganisms may be grown in an aqueous solution, such as wastewater, or by providing a substrate, such as in a reactor comprising a carbon source, such as glucose, fructose, saccharoses, lactose, polysaccharides, such as starch, celluloses, pectin, chitin, and pentosanes, and/or linear or branched carboxylic acids, such as C₁-C₆ carboxylic acids, such as formic acid, acetic acid, propionic acid, butyric acid, valeric acid, and caproic acid, and/or linear or branched alkanols, such as C₁-C₆ alkanols, such as methanol, ethanol, propanol, and butanol, and/or a phosphorus source, and/or a nitrogen source, and combinations thereof. In an example acetate was provided, such as in a mineral medium, such that sialic acids are synthesized by microbes present, i.e. mainly accumulibacter species.

In an exemplary embodiment of the present method a temperature may be maintained between 15-40° C., such as between 20-30° C.

In an exemplary embodiment of the present method a COD may be 200-500 mg/l, preferably 250-400 mg/l, such as 300-350 mg/l.

In an exemplary embodiment of the present method a N content is 40-100 mg/l, preferably 50-80 mg/l, such as 60-75 mg/l.

In an exemplary embodiment of the present method a P content is 1-20 mg/l, preferably 2-15 mg/l, such as 5-10 mg/l.

In an exemplary embodiment of the present method a S content is 1-20 mg/l, preferably 2-15 mg/l, such as 5-10 mg/l.

In an exemplary embodiment of the present method a Cl content is 1-20 mg/l, preferably 2-15 mg/l, such as 5-10 mg/l.

In an exemplary embodiment of the present method a Mg content is 1-20 mg/l, preferably 2-15 mg/l, such as 5-10 mg/l.

In an exemplary embodiment of the present method a pH may be 6-8, preferably 6.5-7.5, such as 6.8-7.2. The pH may be adapted by adding HCl or NaOH, respectively.

In an exemplary embodiment of the present method a dissolved oxygen concentration may be 10-60%, preferably 25-50%, more preferably 30-40%, such as 34-37%.

In an exemplary embodiment of the present method a sludge retention time may be 10-50 days, preferably 15-30 days, such as 18-22 days.

In an exemplary embodiment of the present method an aerobic phase may be 30-120 minutes/cycle, preferably 45-90 minutes/cycle, such as 50-75 minutes/cycle.

In an exemplary embodiment of the present method an anaerobic phase may be 100-360 minutes/cycle, preferably 120-240 minutes/cycle, such as 150-200 minutes/cycle.

In an exemplary embodiment of the present method a settling time may be 1-10 minutes per cycle, preferably 2-7 minutes/cycle, such as 3-6 minutes/cycle.

In an exemplary embodiment of the present method an effluent withdrawal time may be 1-10 minutes per cycle, preferably 2-7 minutes/cycle, such as 3-6 minutes/cycle.

In an exemplary embodiment of the present method the microorganisms may be selected from Proteobacteria, such as Acidithiobacillia, Aiphaproteobacteria, Betaproteobacteria, such as chemolithoautotrophs, photoautotrophs, and heterotrophs, Deltaproteobacteria, Epsilonproteobacteria, Gammaproteobacteria, Hydrogenophilalia, Oligoflexia, such as Ca. Accumilibacter, and fimbria comprising bacteria.

In an exemplary embodiment of the present method microorganisms may be grown in a batch reactor, a continuous reactor, a semi-continuous reactor, such as a Nereda® reactor.

In an exemplary embodiment of the present method the produced granular sludge is incubated at an increased pH of 10-13, such as by adding NaOH. In an alternative one of an alcohol, such as methanol or ethanol, acetone, ammonia, OCl⁻, Cl₂, H₂O₂, and a compound with at least two amine groups, such as urea, may be used.

In an exemplary embodiment the present method may comprise stirring the mixture, such as during 2-10 hours at 200-500 rpm.

In an exemplary embodiment the present method may comprise removing insoluble substances,

In an exemplary embodiment the present method may comprise lowering the pH to 4-6, such as by adding HCl, thereby precipitating extracellular polymeric substance (EPS).

In an exemplary embodiment the present method may comprise adding a water activity lowering compound, such as acetone and isopropylalcohol, and therewith precipitating EPS.

In an exemplary embodiment the present method may comprise freeze drying the precipitate,

In an exemplary embodiment the present method may comprise solubilizing the EPS in an alkaline aqueous solution, such as by using NaOH,

In an exemplary embodiment the present method may comprise denaturating the EPS, such as at a temperature of 60-80° C. during 20-45 minutes.

In an exemplary embodiment the present method may comprise providing enzymes for enzymatic hydrolysis of extracted EPS and separating proteins, such as trypsin, papain and proteinase K enzymes, preferably papain, at elevated temperature, such as at a temperature of 50-80° C., during 10-15 hours, at a pH of 5-8.

In an exemplary embodiment of the present method 0.1-20 wt. % biomedical compound may be extracted, preferably 0.2-15 wt. %, more preferably 1-14 wt. %, such as preferably 2-12 wt. %, wherein wt. % are relative to a total mass of the extracellular matrix. Remarkably both monosaccharides and disaccharides can be extracted, both in relatively large quantities.

In an exemplary embodiment of the present method 0.1-10 wt. % monosaccharide may be extracted preferably 0.2-8 wt. %, more preferably 1-7 wt. %, such as preferably 2-6 wt. %. The monosaccharide may be a sialic acid compound, or the like.

In an exemplary embodiment of the present method 0.1-10 wt. % disaccharide may be extracted, wherein wt. % are relative to a total mass of the extracellular matrix. The disaccharide may be a heparin (such as 5 kDa, 8 kDa, and 12 kDa) compound, or the like.

In a second aspect the present invention relates to a product obtained by the present method, optionally comprising trace compounds of the microorganism culture, such as DNA, and proteins.

In an exemplary embodiment of the method of transferring genes the genes encode GlcNAc-6-P 2-epimerase, Neu5Ac synthetase, CMP Neu5Ac synthetase, and Sialyl transferase. Examples of the genes are GlcNAc-6-P 2-epimerase (of Neisseria meningitides, accession number ABW08136.1), Neu5Ac synthetase (of Chitinivibrio alkaliphilus; ERP39285.1), Neu5Ac synthetase (of Psychrobacter arcticus, WP 011279946.1), Neu5Ac synthetase (of Drosophila melanogaster, NP 650195.1), Neu5Ac synthetase (of Salinibacter ruber, CBH23620.1), CMP Neu5Ac synthetase (of Campylobacter jejuni, AOW97441.1), CMP Neu5Ac synthetase (of Helicobacter cetorum, AFI04478.1), Sialyl transferase (of Halanaerobium praevalens, AD076488.1), and Sialyl transferase (of Photobacterium damselae, BAA25316.1)

The one or more of the above examples and embodiments may be combined, falling within the scope of the invention.

### FIGURES

FIG. 1a-f shows disaccharide units of heparin.

FIG. 2a-d show sialic acids.

FIG. 3 shows images of SDS-PAGE gels.

FIGS. 4a-b show structural formulas of the present monosaccharide and disaccharide.

### DETAILED DESCRIPTION OF THE FIGURES

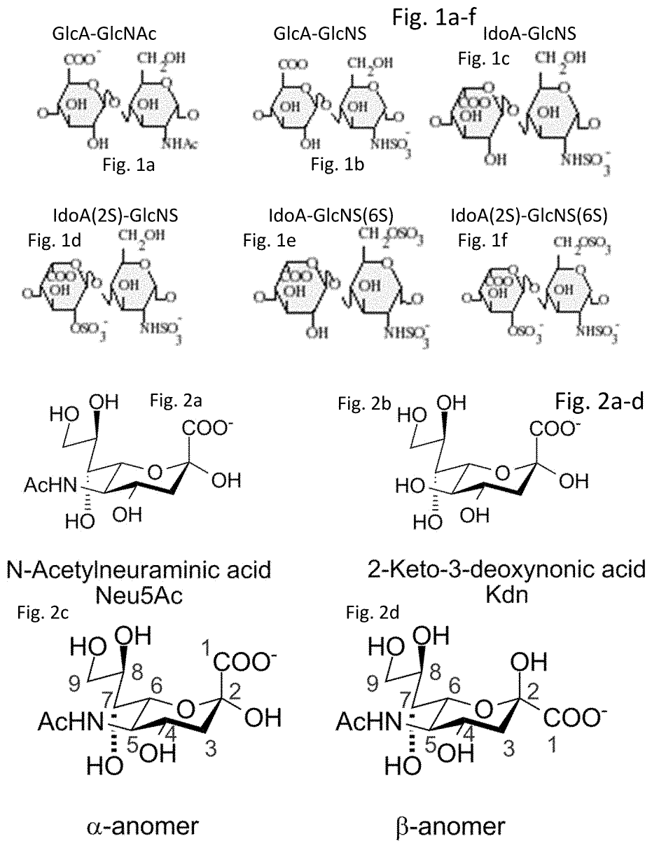

FIG. 1a-f show GlcA=β-D-glucuronic acid, IdoA=α-L-iduronic acid, IdoA(2S)=2-O-sulfo-α-L-iduronic acid, GlcNAc=2-deoxy-2-acetamido-α-D-glucopyranosyl, GlcNS=2-deoxy-2-sulfamido-α-D-glucopyranosyl, GlcNS(6S)=2-deoxy-2-sulfamido-α-D-glucopyranosyl-6-O-sulfate.

FIG. 2a-d show N-acetylneuraminic acid (Neu5Ac), 2-keto-3-deoxynonic acid Kdn, an α-anomer, and a β-anomer.

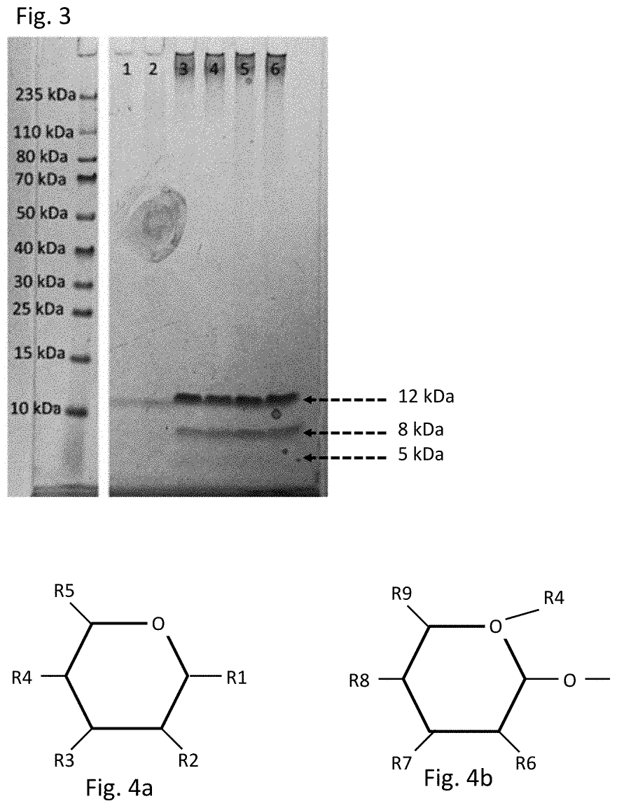

FIG. 3 shows Images of SDS-PAGE gels, showing 12 kDa and 8 kDa bands. On the left (A) stained with Alcian Blue pH 2.5. On the right (B) stained with PAS. The same numbering applies as to FIG. 1; Lane 1: Dye-sGAG complex from Blyscan assay of untreated Anammox, lane 2: Dye-sGAG complex from blyscan assay of denatured Anammox hydrolysed with Papain, lane 3: untreated Anammox, lane 4: Denatured Anammox, lane 5: Anammox hydrolysed with Trypsin, lane 6: Denatured Anammox, hydrolysed with Trypsin. FIGS. 4a-b have been detailed above.

The invention is further detailed by the accompanying examples, which is exemplary and explanatory of nature and are not limiting the scope of the invention. To the person skilled in the art it may be clear that many variants, being obvious or not, may be conceivable falling within the scope of protection, defined by the present claims.

### EXAMPLE

### Reactor Operation and Dominant Microorganisms

Reactor Operation

Seawater-adapted aerobic granular sludge was cultivated in a 2.7 L bubble column (5.6 cm diameter), operated as a sequencing batch reactor (SBR). The reactor was inoculated with Nereda® sludge. The temperature was controlled at 20° C., the pH at 7.0±0.1, dissolved oxygen (DO) concentration at 50% saturation. The average sludge retention time (SRT) was 20 days, and reactor cycles related to 60 minutes of anaerobic feeding, 170 minutes aeration, 5 minutes settling and 5 minutes effluent withdrawal. A feed of 1.5 L per cycle consisted of 1200 mL of artificial seawater (Instant Ocean®, final concentration 35 g/L), 150 mL of medium A, and 150 mL of medium B. Medium A contained 7.785 g/L sodium acetate trihydrate (3.66 g/L COD), 0.88 g/L MgSO₄.7H₂O, and 0.35 g/L KCl. Medium B contained 2.289 g/L NH₄Cl (600 mg/L NH₄^{+—N),} 349 mg/L K₂HPO₄, and 136 mg/L KH₂PO₄. The combination of these feed streams led to influent concentrations of 366 mg/L COD, 60 mg/L NH₄⁺—N and 9.3 mg/L PO₄³⁻—P. Acetate was completely consumed anaerobically within the first 60 minutes of the cycle, while phosphate was released up to 75 mg P043-P/L (5.9 net P-mol release). This corresponds to 0.34 P-mol/C-mol of anaerobic phosphate release per carbon uptake. Fluorescence in situ hybridization (FISH) analysis was performed for microbial community analysis. Probes for polyphosphate accumulating organisms (PAOmix), glycogen accumulating organisms (GAOmix), and a general probe for all bacteria (EUB338) were used. The results indicate dominance of PAO over GAO in the present system.

Commercially available lectins (FITC or Alexa488) were applied as an individual probe to one granule. After this glycoconjugates screening, granules were stained specifically for proteins and sialic acids. The result of lectin staining showed that sialic acid is abundantly distributed in granular sludge. To quantify the amount of sialic acids, neuraminidase was applied that cleave α(2→3,6,8,9) N-acetylneuraminic acid linkages, as well as branched N-acetylneuraminic acid. Subsequent quantification yields an amount of 11.33±3.80 mg N-acetylneuraminic acid per gram of volatile solids (VS).

Quantification of sialic acid (neuraminic acid, Neu5Ac) in the seawater-adapted AGS was performed with a Sialic Acid Quantitation Kit (Sigma-Aldrich, USA). The protocol was followed as described in the manual that was supplied with the quantitation kit for a whole cell assay. 80 μL of homogenized cells were taken, added to 20 μL sialidase buffer and 1 μL of α(2→3,6,8,9)-neuraminidase, and incubated overnight at 37° C. Afterwards, 20 μL 0.01M β-NADH solution, 1 μL of N-Acetylneuraminic Acid Aldolase and 1 μL of Lactic Dehydrogenase were added, and incubated at 37° C. for 1 hour. Absorbance at 340 nm was measured prior and after addition of the last enzymes and used for calculation of the Neu5Ac concentration.

Typically the sialic acid, and likewise the heparin-like compound can be obtained in relatively pure form. Typically purities found are >50%, such as >60%.

### Genome Transferase

The species from which the reference protein sequences were taken were a range of pathogenic bacteria (Neisseria meningitides, Campylobacter jejuni, Helicobacter cetorum, Photobacterium damselae), extremophiles (Chitinivibrio alkaliphilus, Psychrobacter arcticus, Salinibacter ruber, Halanaerobium praevalens), and the common fruit fly (Drosophila melanogaster). The criteria for low E-value, and thereby high probability for presence in its genome, are set at <1E-40.

Glycosaminoglycan Extraction

Anammox granules and AGS were obtained from commercially operated reactors in Sluisjesdijk and Dinxperlo respectively. The AGS reactor was operated as described above. The anammox reactor was operated as follows. A full-scale anammox reactor of 70 m³ was used. The reactor combines a high loading rate with efficient biomass retention, characteristics which the anammox process has in common with anaerobic wastewater treatment. The lower compartment (ca 40 m³) is mixed by influent and down-corner flow as well as by gas recycled from the top of the reactor. On top of the lower compartment, gas is collected for the riser of the gas lift. The liquid moves from the lower compartment to the less mixed and thus stratified upper compartment, serving mainly for biomass retention and effluent polishing. The feed is introduced from the bottom of the reactor and is (during loads lower than ca 8 m³/h or 150 kg-N/d) mixed with an additional recirculation flow from the effluent of the reactor to maintain adequate up flow velocity and shear stress to favour granule formation.

The design load was 500 kg-N/d (7.1 kg-N/m³/d) but the practical maximum loading is determined by the amount of nitrogen in the sludge digestate (on average ca 700 kg-N/d). At the sludge treatment site sludge is thickened and digested (residence time ca 30 days, temperature 32-33° C.). The start-up involved two phases. The start-up regime was characterized by a relatively high influent flow rate (on average 3.6 m³/h, HRT ¼ 19.4 h) with a low concentration of nitrite (on average 120 mg-N/1). During the start-up of the anammox reactor, the aim was to produce an effluent containing nitrite at non-toxic levels. An additional economical advantage of this mode of operation was that the nitrogen removal of the sludge treatment as a whole remained high during this phase in the start-up of the anammox reactor. In the second part of the start-up, methanol dosing to the nitritation reactor was completely stopped and the reactor was running as a nitritation reactor with nitrite effluent concentrations close to 600 mg-N/l. The nitrite:ammonium ratio of circa 1:1—which is required for the anammox process was obtained automatically. A flow-adjustable recycle stream from the top of the reactor was mixed with the influent to maintain a sufficiently high up flow rate (2-3 m/h) during the phases in the start-up when the influent rate. After the reactor was converting at its design capacity of 500 kg-N/d, sludge was removed periodically from the bottom of the reactor. A total amount of 36 m³ of sludge was removed in amounts varying from 0.5 to 2 m³.

The extracted EPS's were pre-treated in line with a general method to prepare proteins before they are used in mass-spectrometry (MS) (ThermoFischer). Enzyme hydrolysis (trypsin, papain, proteinase K), dimethylmethylene blue assay (DMMB), and sodium dodecyl sulphate polyacrylamide gel electrophoresis we sequentially used to prepare samples. Glycoproteins were stained using the periodic acid-Schiff (PAS) method, Pierce™ glycoprotein staining kit. For negatively charged glycans Alcian Blue 8GX (Sigma) was used at pH 2.5. Sulphated glycans were stained using Alcian Blue at pH 1.0. Initial results showed that AGS contains around 4.5 μg sGAG per mg dry weight and Anammox around 0.33 μg sGAG per mg dry weight for the DMMB assay. A first optimization led to increased measured sGAG content (1.2% for Anammox EPS and 6.4% for AGS EPS). The annamox sGAG content was found to be between 1.4% and 2.0% (w/w) after denaturation. It was found that protein digestion and denaturation led to an increased amount of sGAG content. For annamox a concentration between 1.2% and 2.6% was found, whereas for AGS a concentration between 5.5% and 6.7% was found (w/w).

Obtained sGAG's had typical weights of 10-15 kDa, such as about 12 kDa. Also 8 kDa and 5 kDa sGAG's were found.

1. A method of producing a biomedical compound, comprising
providing a microorganism culture,
growing the microorganisms under aerobic and anaerobic conditions by switching at least once between aerobic and anaerobic conditions thereby favouring carbon accumulating microorganisms comprising PAOs (poly-phosphate accumulating organisms) and GAOs (glycogen accumulating organisms),
forming an extracellular matrix embedding microorganisms, the matrix comprising extracellular polymeric substances,
physically separating the extracellular matrix embedding the microorganisms, and
extracting the biomedical compound from the extracellular matrix, wherein the biomedical compound comprises at least one of a monosaccharide and/or disaccharide and is selected from (i) at least one of a heparan like polymer, a heparin like compound, and a heparin oligomer, and from (ii) at least one of a sialic acid, a glycoprotein, and a glycolipid, or a salt thereof, or conjugate thereof, or a combination thereof.

2. The method according to claim 1, wherein the biomedical compound is selected from a 3-30 kDa glycosaminoglycan.

3. The method according to claim 1, wherein the monosaccharide or disaccharide of the biomedical compound has structural formula 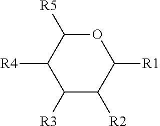
wherein each of R1-R5 is independently selected,
wherein R1 is selected from at least one of H, COOH, and OH,
wherein R2 is selected from at least one of NHAc, NHSO3H, and H,
wherein R3 is selected from at least one of H, and OH,
wherein R4 is selected from at least one of H, NHAc, OH, and 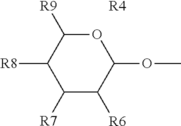
wherein R6 is selected from at least one of H, OSO3H, and OH,
wherein R7 is selected from at least one of OH, and H,
wherein R8 is selected from at least one of H, and OH,
wherein R9 is selected from at least one of H, COOH, and OH, and
wherein R5 is selected from at least one of H, CH2OH, CH2OSO3, COOH, CHOHCH2OH, CH2CHOHCH2OH, and OH.

4. The method according to claim 1, wherein the microorganisms are grown in granules.

5. The method according to claim 1, wherein the microorganisms form aerobic granular sludge, or wherein the microorganisms are grown under aerobic and anaerobic conditions by switching between aerobic and anaerobic conditions in a cyclic mode.

6. The method according to claim 1, wherein the compound is a 5-15 kDa glycosaminoglycan.

7. The method according to claim 1, wherein the compound is a sialic acid.

8. The method according to claim 1, wherein the microorganisms are provided with a supplement comprising at least one of sugars, fatty acids, proteins, proteins, and minerals.

9. The method according to claim 1, wherein the microorganisms are grown in an aqueous solution, or by providing a substrate, in a reactor comprising a carbon source and linear or branched carboxylic acids, and linear or branched alkanols, and a phosphorus source, and a nitrogen source, and combinations thereof.

10. The method according to claim 1,
wherein a temperature is maintained between 15-40° C., and
wherein a COD is 200-500 mg/l, and
wherein a N content is 40-100 mg/1, and
wherein a P content is 1-20 mg/1, and
wherein a S content is 1-20 mg/1, and
wherein a Cl content is 1-20 mg/1, and
wherein a Mg content is 1-20 mg/1, and
wherein a pH is 6-8, and
wherein a dissolved oxygen concentration is 10-60%, and
wherein a sludge retention time is 10-50 days, and
wherein an aerobic phase is 30-120 minutes/cycle, and
wherein an anaerobic phase is 100-360 minutes/cycle, and
wherein a settling time is 1-10 minutes per cycle, and
wherein an effluent withdrawal time is 1-10 minutes per cycle.

11. The method according to claim 1, wherein the microorganisms are selected from Proteobacteria, Alphaproteobacteria, Betaproteobacteria, Deltaproteobacteria, Epsilonproteobacteria, Gammaproteobacteria,
Hydrogenophilalia, Oligoflexia, and fimbria comprising bacteria.

12. The method according to claim 1, wherein microorganisms are grown in a batch reactor, a continuous reactor, or a semi-continuous reactor.

13. The method according to claim 1, wherein the produced granular sludge is incubated at increased pH of 9-12,
stirring the mixture,
removing insoluble substances,
lowering the pH to 4-6,
freeze drying the precipitate,
solubilizing the EPS in an alkaline aqueous solution,
optionally reducing sulphide bridges,
denaturating the EPS, at a temperature of 60-80° C. during 20-45 minutes, and
providing enzymes for enzymatic hydrolysis of extracted EPS and separating proteins, at elevated temperature, at a temperature of 50-80° C., during 10-15 hours, at a pH of 5-8.

14. The method according to claim 1, wherein 0.1-20 wt. % biomedical compound is extracted, wherein wt. % are relative to a total mass of the extracellular matrix.

15. The method according to claim 1, wherein 0.1-10 wt. % monosaccharide is extracted, and wherein 0.1-10 wt. % disaccharide is extracted, wherein wt. % are relative to a total mass of the extracellular matrix.

16. A product obtained by claim 1, comprising 0.1-30 wt. % heparan like polymer, a heparin like compound, and a heparin oligomer is/are present, and 0.1-30 wt. % a neuraminic acid glycosaminoglycan, a sialic acid, a glycoprotein, and a glycolipid, and optionally comprising trace compounds of the microorganism culture.

17. (canceled)

18. (canceled)
